(19) United States
(12) Patent Application Publication
(10) Pub. No.: US 2002/0027204 A1
(43) Pub. Date: Mar. 7, 2002

(54) Electron beam exposure apparatus, device for shaping a beam of charged particles and method for manufacturing the device

(75) Inventors: Harunobu Muto, Tokyo (JP); Hiroshi Yano, Tokyo (JP)

Correspondence Address: David L. Fehrman, Morrison & Foerster LLP, 35th Floor, 555 W. 5th Street, Los Angeles, CA, 90013, US

(73) Assignee: ADVANTEST CORPORATION

(21) Appl. No.: 09/946,395

(22) Filed: Sep. 4, 2001

(30) Foreign Application Priority Data
Sep. 4, 2000 (JP) 2000-266742

(51) Int. Cl. G21G 5/00; A61N 5/00

(52) U.S. Cl. 250/492.3

(57) ABSTRACT

The present invention provides a device including a first channel formed on a substrate of the device, the first channel including a pair of substantially parallel sides; and a second channel formed on the substrate of the device, the second channel including a pair of parallel sides substantially perpendicular to and overlapped with the pair of substantially parallel sides of the first channel, wherein the opening perforates the substrate of the device and is formed at an area defined by the overlapped pairs of sides of the first and second channels.

This patent application claims priority based on a Japanese patent application, 2000-266742 filed on Sep. 4, 2000, the contents of which are incorporated herein by reference.

### BACKGROUND OF THE INVENTION

1. Field of the Invention

The present invention relates to an electron beam exposure apparatus including a device for shaping a beam of charged particles, and a method for manufacturing the device. In particular, the present invention relates to a device for forming a beam of charged particles into a desirable and precise rectangular cross-sectional shape.

2. Description of the Related Art

FIG. 1 shows an example of a conventional device 500 having an opening area 506 for adapting a cross-sectional shape of an electron beam. The device 500 includes a pair of blades or strips 502 provided approximately parallel to each other, and another pair of blades or strips 504 provided approximately perpendicular to the blades 502. The cross-sectional shape of an electron beam is formed into a rectangular shape by the pairs of blades 502 and 504 while the beam is illuminated through the opening 506 of the device 500.

The conventional device 500 used to form the cross-sectional shape of the electron beam is manufactured by a precision machine manufacturing technology. However, in recent years, with miniaturization of electronic devices, such as semiconductor devices, the cross-sectional shape of the electron beam of, for example, an electron beam exposure apparatus is required to be formed into a highly precise and minute rectangular shape. Accordingly, it is very difficult to manufacture the device 500 by the conventional precision machine manufacturing technology. Further, recently, an electron beam exposure apparatus using a plurality of electron beams is under development. However, by using the conventional precision machine manufacturing technology, it is extremely difficult to provide a plurality of opening areas, used for forming the cross-sectional shapes of the plurality of electron beams into rectangles, at predetermined locations of the device 500 with high precision. Therefore, it is almost impossible to practically or commercially use the aforementioned electron beam exposure apparatus.

### SUMMARY OF THE INVENTION

Therefore, it is an object of the present invention to provide a device for shaping a beam of charged particles and a method for manufacturing the device which overcomes the above issues in the related art. This object is achieved by combinations described in the independent claims. The dependent claims define further advantageous and exemplary combinations of the present invention.

According to the first aspect of the present invention, a device comprising an opening for shaping a beam of charged particles to provide a desired cross-sectional shape thereof, comprising: a first channel formed on a substrate of the device, the first channel including a pair of substantially parallel sides; and a second channel formed on the substrate of the device, the second channel including a pair of parallel sides substantially perpendicular to and overlapped with the pair of substantially parallel sides of the first channel, wherein the opening perforates the device and is formed at an area defined by the overlapped pairs of sides of the first and second channels.

The second channel may be formed on an opposite side of the substrate of the device than a side thereof where the first channel is formed.

A distance between the pair of sides of the first channel may be substantially the same as a distance between the pair of sides of the second channel. Also, more than one of the openings perforating the device may be formed.

According to the second aspect of the present invention, a device comprising an opening having a cross-sectional shape for shaping a beam of charged particles, comprising: a base having a hole formed therein, the hole having a first pair of substantially parallel sides and a second pair of parallel sides which are substantially perpendicular to the first pair of substantially parallel sides of the hole; and an inscribed element formed to contact with an inside surface of the hole, wherein the inscribed element has an opening formed inside the hole, the opening of the inscribed element perforating the device and including vertexes that are sharper than corresponding vertexes of the hole.

According to the third aspect of the present invention, a method for manufacturing a device comprising an opening for shaping a beam of charged particles to provide a desired cross-sectional shape thereof, comprising: forming a first layer having a first hole on a substrate, the first hole having a pair of substantially parallel sides; forming a second layer having a second hole on the first layer, the second hole having a pair of parallel sides substantially perpendicular to and overlapped with the pair of substantially parallel sides of the first hole, wherein the opening perforates the device and is formed at an area defined by the overlapped pairs of sides of the first and second holes; and separating the substrate.

Forming the first layer may comprise: forming a first resist pattern on an area of the substrate where the first hole is formed; and selectively forming the first layer on the substrate; and the forming the second layer comprises: forming a second resist pattern on an area of the first layer and the first resist pattern where the second hole is formed; and selectively forming the second layer on the first layer.

The substrate may be made of a conductive material, and the first layer and the second layer may be formed by electrode position.

The first layer may be formed to be thicker than the first resist pattern, and the second layer may be formed to be thicker than the second resist pattern.

The method may further comprise: separating the first layer from the substrate, wherein the second layer is formed on a surface of the first layer with which the substrate originally contacted.

According to the fourth aspect of the present invention, a method for manufacturing a device with an opening having a cross-sectional shape for shaping a beam of charged particles, comprising: forming a first channel having a first pair of substantially parallel sides on a base; and forming a second channel on the base, the second channel having a second pair of parallel sides substantially perpendicular to and overlapped with the first pair of substantially parallel sides of the first channel, wherein the opening perforates the device and is formed at an area defined by the overlapped first and second pairs of sides of the first and second channels.

The second channel may be formed on a side of the base that is opposite to another side of the base on which the first channel is formed.

According to the fifth aspect of the present invention, a method for manufacturing a device comprising an opening for shaping a beam of charged particles to have a selected cross-sectional shape, comprising: forming a base having a hole formed therein, the hole being defined by a first pair of substantially parallel sides and a second pair of parallel sides substantially perpendicular to the first pair of substantially parallel sides of the hole; and forming an inscribed element in the hole to contact with an inside surface of the hole, wherein the inscribed element has an opening formed inside the hole, the opening of the inscribed element perforates the device and includes vertexes that are sharper than corresponding vertexes of the hole.

According to the sixth aspect of the present invention, an electron beam exposure apparatus for exposing an electron beam on a desired area of a wafer, comprising: an electron gun for generating the electron beam; an electron lens for adjusting focus of the electron beam; a deflector for deflecting the electron beam on a desired area of a wafer; a device for shaping the electron beam to have a predetermined cross-sectional shape; and a wafer stage for supporting a wafer, wherein the device for shaping the electron beam comprises: a first channel in the device, the first channel having a pair of substantially parallel sides; a second channel in the device, the second channel having a pair of parallel sides substantially perpendicular to and overlapped with the pair of substantially parallel sides of the first channel; and an opening which perforates the device and is formed on an area defined by the overlapped pairs of sides of the first and second channels.

According to the seventh aspect of the present invention, a device for shaping a beam of charged particles, comprising: a first channel formed in the device to have a first pair of substantially parallel sides; a second channel formed in the device to have a second pair of substantially parallel sides, the second pair of sides of the second channel being substantially perpendicular to and overlapped with the first pair of sides of the first channel; and an opening perforating the device, the opening having a substantially rectangular shape defined by the overlap of the first pair and second pair of sides of the first and second channels, wherein a beam of charged particles is passed through the opening to provide a predetermined cross-sectional shape.

This summary of the invention does not necessarily describe all necessary features so that the invention may also be a sub-combination of these described features.

### BRIEF DESCRIPTION OF THE DRAWINGS

FIG. 1 shows an example of a conventional device 500 having an opening area for adapting the cross-sectional shape of an electron beam.

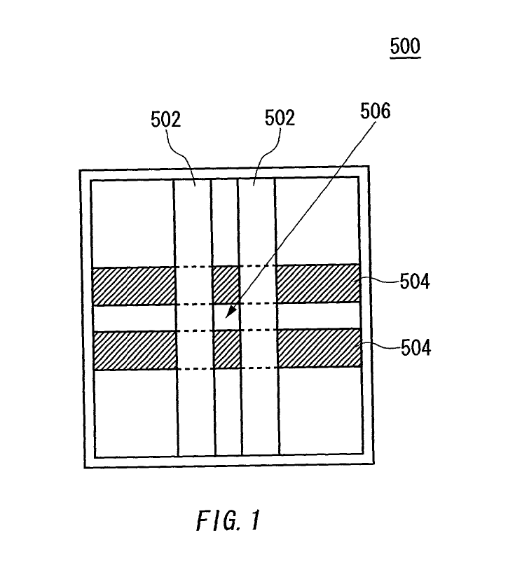

FIG. 2 shows an electron beam exposure apparatus 100 according to an embodiment of the present invention.

FIGS. 3(a) and 3(b) show a device 200, such as the first or second shaping device 14 or 22 in FIG. 2, having a plurality of openings for shaping cross sections of charged particle beams, such as an electron beam.

FIGS. 4(a) to 4(d) show other embodiments of the device 200 having an opening 230.

FIGS. 5(a) to 5(e) show an embodiment of a method for manufacturing the device 200 for shaping a charged particle beam according to the present invention.

FIGS. 6(a) to 6(f) show another embodiment of the method for manufacturing the device 200 according to the present invention.

FIGS. 7(a) to 7(d) show yet another embodiment of the method for manufacturing the device 200 according to the present invention.

FIGS. 8(a) to 8(d) show yet another embodiment of the method for manufacturing the device 200 according to the present invention.

### DETAILED DESCRIPTION OF THE INVENTION

The invention will now be described based on the preferred embodiments, which do not intend to limit the scope of the present invention, but exemplify the invention. All of the features and the combinations thereof described in the embodiment are not necessarily essential to the invention.

As used herein, the term “channel” describes a structure such as a trench, cut, furrow or groove, which is formed in a base or substrate.

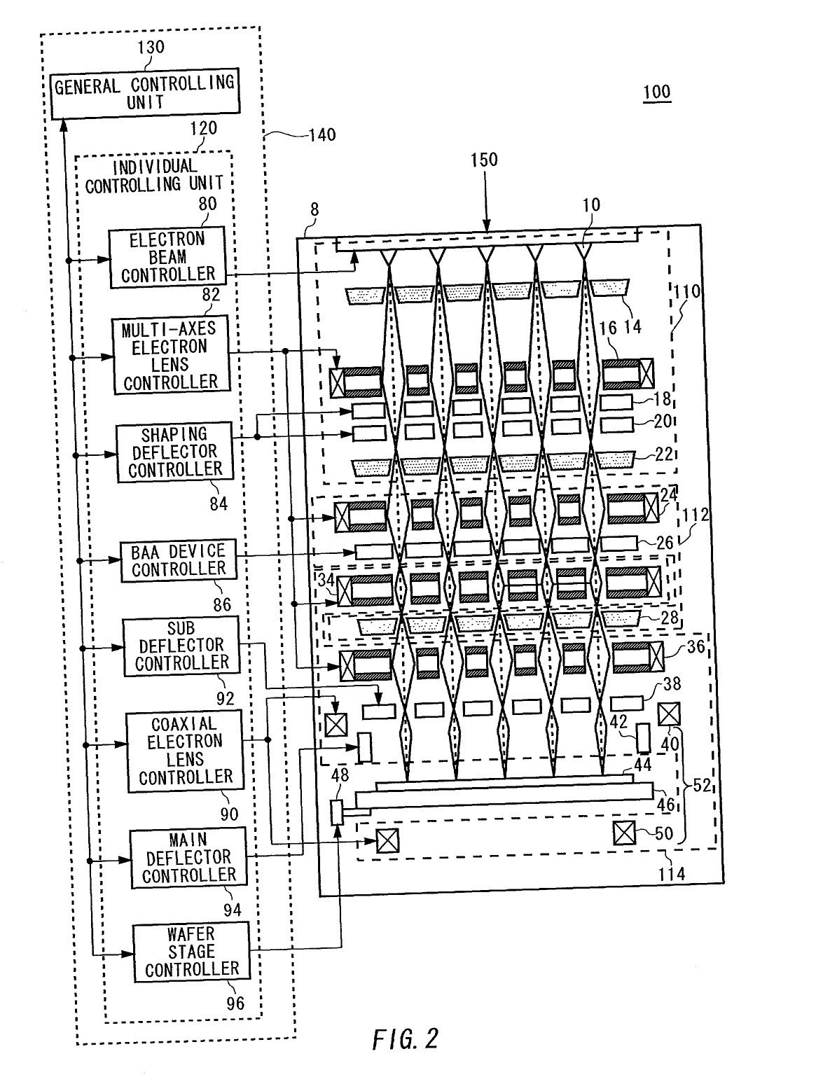

Referring to FIGS. 2 to 8, embodiments of the present invention are described in detail. The embodiments described hereunder should not be construed to be limiting the scope of the present invention defined by the claims, and the features of the present invention described according to the embodiments should not be construed to be essential to practice technical idea of the present invention.

FIG. 2 shows an electron beam exposure apparatus 100 according to an embodiment of the present invention. The electron beam exposure apparatus 100 includes an exposing unit 150 for performing an exposure process of an electron beam on a wafer 44 and a control section 140 for controlling the operation of elements included in the exposing unit 150.

The exposing unit 150 includes an electron beam shaping unit 110 for generating a plurality of electron beams and shaping the cross sections of the electron beams into desired shapes, an exposure switching unit 112 for independently switching an electron beam according to an independent determination as to whether or not the electron beam is to be exposed on the wafer 44, and a projecting unit 114 for adjusting direction and size of a pattern which is transcribed on the wafer 44. The exposing unit 150 further includes a stage section having a wafer stage 46 for supporting the wafer 44 on which the pattern is transcribed and a wafer stage driver 48 for driving the wafer stage 46.

The electron beam shaping unit 110 includes an electron gun 10 for generating a plurality of electron beams, a first shaping device 14 and a second shaping device 22 respectively having a plurality of openings for shaping cross sections of the electron beams into desired shapes by passing the electron beams through the openings, a first multi-axes electron lens 16 for adjusting a focus of an electron beam by independently concentrating respective electron beams, and a first shaping deflector 18 and a second shaping deflector 20 for independently deflecting respective electron beams passed through the first shaping device 14.

The first shaping device 14 forms the cross-sectional shapes of the electron beams into desired shapes, and has a first channel having two sides, which are approximately parallel to each other, and a second channel, overlapped with the first channel, having two sides approximately perpendicular to the two sides of the first channel. Preferably, an opening through the shaping device 14 is formed at an area defined by the overlapping two pairs of sides of the first and second channels. The second shaping device 22 has the same function and structure as the first shaping device 14.

The first and second shaping devices 14 and 22 may respectively have metal films made of, for example, platinum for grounding the respective surfaces of the first and second shaping devices 14 and 22 on which the electron beams are irradiated. It is preferable to use a high melting point metal material for making the first and second shaping devices 14 and 22 and/or the metal films. Each opening of the first and/or second shaping devices 14 and/or 22 may become wider along the direction of irradiation of the electron beams, as shown in cross section in FIG. 2, in order for the electron beams to effectively pass through corresponding openings.

The exposure switching unit 112 includes a second multi-axes electron lens 24 for adjusting a focus of an electron beam by independently concentrating respective electron beams, a blanking aperture array (“BAA”) device 26 for independently switching an electron beam by deflecting each of the plurality of electron beams according to an independent determination on whether or not the electron beam is to be exposed on the wafer 44, and an electron beam cut off unit 28 having a plurality of openings, through which electron beams are passed, for cutting off an electron beam deflected by the BAA device 26. Each opening of the electron beam cut off unit 28 may become wider along the direction of irradiation of the electron beams, as shown in cross section in FIG. 2, in order for the electron beams to effectively pass through corresponding openings.

The projecting unit 114 includes a third multi-axes electron lens 34 for decreasing a cross-sectional area of an electron beam by independently concentrating respective electron beams, a fourth multi-axes electron lens 36 for adjusting a focus of an electron beam by independently concentrating respective electron beams, a sub deflector 38 for independently deflecting each of the plurality of electron beams onto a predetermined location of the wafer 44, a coaxial lens 52, which functions as an object lens, having a first and a second coil 40 and 50 for concentrating electron beams, a main deflector 42 for concentrating only a desired amount of electron beams to approximately the same direction. The main deflector 42 may be a static electricity type deflector, which deflects a plurality of electron beams at a high speed by using an electric field and is comprised of a circular eight-pole structure having four pairs of poles facing one another, or more than eight (8) poles. The coaxial lens 52 is preferably provided closer to the wafer 44 than to the multi-axes electron lens 36.

The control section 140 includes a general controlling unit 130 and an individual controlling unit 120. The individual controlling unit 120 includes an electron beam controller 80, a multi-axes electron lens controller 82, a shaping deflector controller 84, a BAA device controller 86, a coaxial lens controller 90, a sub deflector controller 92, a main deflector controller 94 and a wafer stage controller 96. The general controlling unit 130 may be, for example, a workstation generally controlling each of the controllers included in the individual controlling unit 120. The electron beam controller 80 controls the electron beam generator 10. The multi-axes electron lens controller 82 controls currents provided to the first, second, third and fourth multi-axes electron lenses 16, 24, 34 and 36.

The shaping deflector controller 84 controls the first and second shaping deflectors 18 and 20. The BAA device controller 86 controls voltage applied to a deflection electrode included in the BAA device 26. The coaxial lens controller 90 controls currents provided to the first and second coils 40 and 50 included in the coaxial lens 52. The main deflector controller 94 controls voltage applied to the deflection electrode included in the main deflector 42. The wafer stage controller 96 controls the wafer driver 48 to move the wafer stage 46 to a predetermined location.

Now, the operation of the electron beam exposure apparatus 100 of the present invention is described in detail. First, the electron gun 10 generates a plurality of electron beams. The electron beams generated by the electron beam generator 10 are irradiated and shaped by the first shaping device 14. The respective electron beams passed through the first shaping device 14 have rectangular cross sections according to the openings of the first shaping device 14.

Each of the electron beams having rectangular cross section is independently concentrated by the first multi-axes electron lens 16, and focus of each of the electron beams is independently adjusted in regard to the second shaping device 22 by the first multi-axes electron lens 16. Each of the plurality of electron beams having rectangular cross section is independently deflected to a desired location on the second shaping device 22. Each of the electron beams, deflected by the first shaping deflector 18, is independently and vertically deflected in regard to the second shaping device 22 by the second shaping deflector 20. The above-described operations of the first multi-axes electron lens 16 and the first shaping deflector 18 are performed on each one of the electron beams independently of other electron beams; in other words, operations of the first multi-axes electron lens 16 and the first shaping deflector 18 are independently performed on each one of the electron beams such that the respective electron beams do not affect each other or are not affected by operations performed on other electron beams. As a result, the electron beams are controlled to be vertically irradiated on the second shaping device 22 at desired locations of the second shaping device 22. The second shaping device 22 having a plurality of rectangular openings further shapes the plurality of electron beams, which are irradiated to the openings of the second shaping device 22 and have rectangular cross sections, into more desirable and precise rectangular cross sections, so that the electron beams are suitable to be irradiated on the wafer 44.

By the second multi-axes electron lens 24, each of the electronic beams is concentrated independently of one another, and focus of each of the electron beams is independently adjusted in regard to the BAA device 26. Each of the electron beams, of which the focus is adjusted by the second multi-axes electron lens 24, passes through each of a plurality of apertures included in the BAA device 26.

The BAA device controller 86 determines whether or not a voltage is applied to each of the deflection electrodes provided at a place near to each of the apertures included in the BAA device 26. The BAA device 26 controls irradiation of each of the electron beams to the wafer 44 based on the voltage applied to each of the deflection electrodes. In case the voltage is applied, an electron beam passed through the aperture is not irradiated on the wafer 44 because the electron beam is deflected and can not pass through an opening included in the electron beam cut off unit 28. In case the voltage is not applied, an electron beam passed through the aperture is irradiated on the wafer 44 because the electron beam is not deflected and can pass through an opening included in the electron beam cut off unit 28.

Cross-sectional area of an electron beam not deflected by the BAA device 26 is decreased by the third multi-axes electron lens 34 and passes through the opening included in the electron beam cut off unit 28. By the fourth multi-axes electron lens 36, the plurality of electron beams are independently concentrated, and foci of the electron beams are independently adjusted in regard to the sub deflector 38. The electron beams, of which foci are adjusted, are irradiated into deflecting elements included in the sub deflector 38.

The plurality of deflecting elements included in the sub deflector 38 are independently controlled by the sub deflector controller 92. The plurality of electron beams, irradiated into the deflectors of the sub deflector 38, are independently deflected to desired exposure locations on the wafer 44 by the sub deflector 38.

During an exposure process, the wafer stage controller 96 controls the wafer stage driver 48 so that the wafer stage 46 is moved in a predetermined direction. The BAA device controller 86 decides an aperture through which an electron beam passes based on exposure pattern data, and performs power controls on each of the apertures. In response to the movement of the wafer 44, by properly switching apertures through which an electron beam passes, and by deflecting electron beams using the main deflector 42 and sub deflector 38, it is possible to expose desired circuit patterns on the wafer 44.

According to the electron beam exposure apparatus 100, cross 11 section of an electron beam can be formed into a desired rectangular shape. Therefore, for example, in case a wiring pattern having a direct line is exposed, it is possible to have a direct line pattern on the wafer 44 even with an exposure apparatus which irradiates electron beams as pulses. Further, the electron beam exposure apparatus 100 can also be used as a block exposure type or a BAA type apparatus.

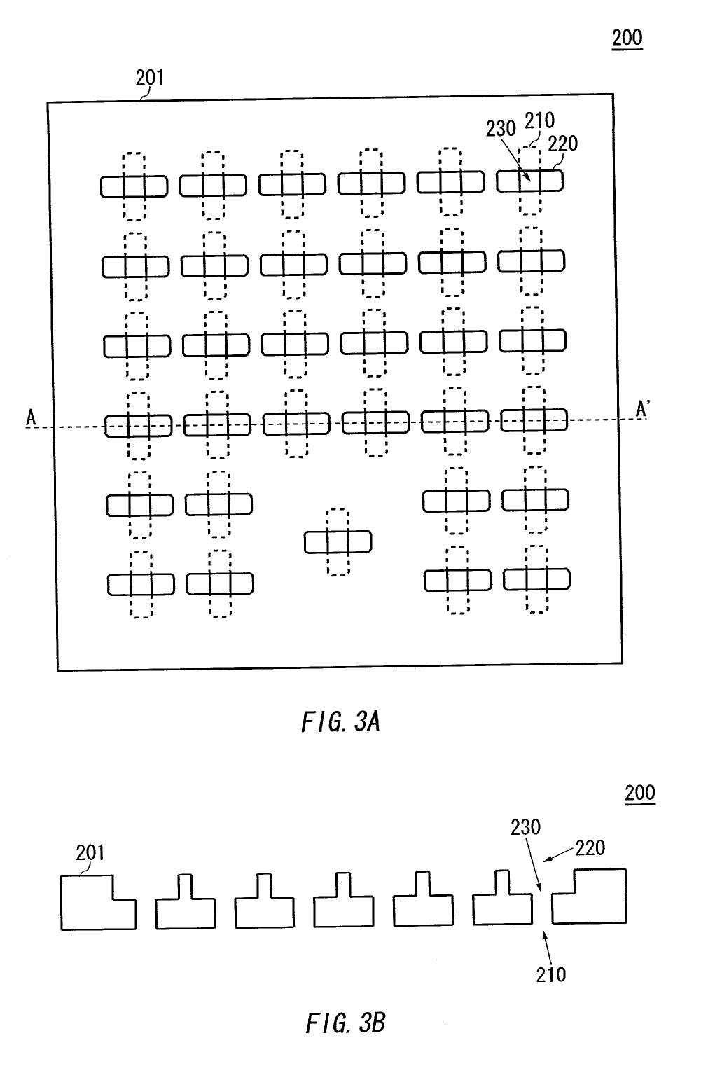

FIGS. 3(a) and 3(b) show a device 200, such as the first or second shaping device 14 or 22, having a plurality of openings for shaping cross sections of charged particle beams, such as electron beam. FIGS. 3(a) and 3(b) are a plane view and a cross-sectional view seen from line A-A′ of the device 200, respectively.

As shown in FIGS. 3(a) and 3(b), the device 200 has a substrate 201 on which a first channel 210 having two sides approximately parallel to each other and a second channel 220, overlapped with the first channel 210, having two sides approximately perpendicular to the two sides of the first channel 210 are formed. Further, an opening 230 is formed on an area of the substrate 201 defined by the two pairs of sides of the first and second channels 210 and 220. Therefore, an irradiated electron beam is shaped into a desired cross section by passing through the opening 230. Concretely, the cross section of the passed electron beam has a shape corresponding to the shape of the opening 230 or the shape of the vertexes of the opening 230.

As shown in FIG. 3(b), it is preferable to form the second channel 220 on an opposite side of the substrate 201 than the side where the first channel 210 is formed. Further, it is preferable to make a distance between the two sides of the first channel 210 substantially the same with a distance between the two sides of the second channel 220. Concretely, it is preferable for the opening 230 to be substantially a square when seen from the side of the electron beam generator 10. According to another embodiment of the present invention, the shape of the opening 230 may be a rectangle other than a square.

According to the present invention, the device 200, which forms the cross-sectional shape of the charged particle beams, may have an opening of substantially right-angled vertexes by including the first and second channels 210 and 220. Therefore, it is possible to form a charged particle beam of rectangular cross section having substantially right-angled vertexes. Further, since it is possible to form the opening 230 into a minute rectangle, it is possible to form a plurality of openings 230 at desired locations on the device 200 with very high precision and ease. Further, it is still possible to simultaneously form a plurality of devices 200 on which a plurality of openings 230 of desired shapes are formed at respective desired locations.

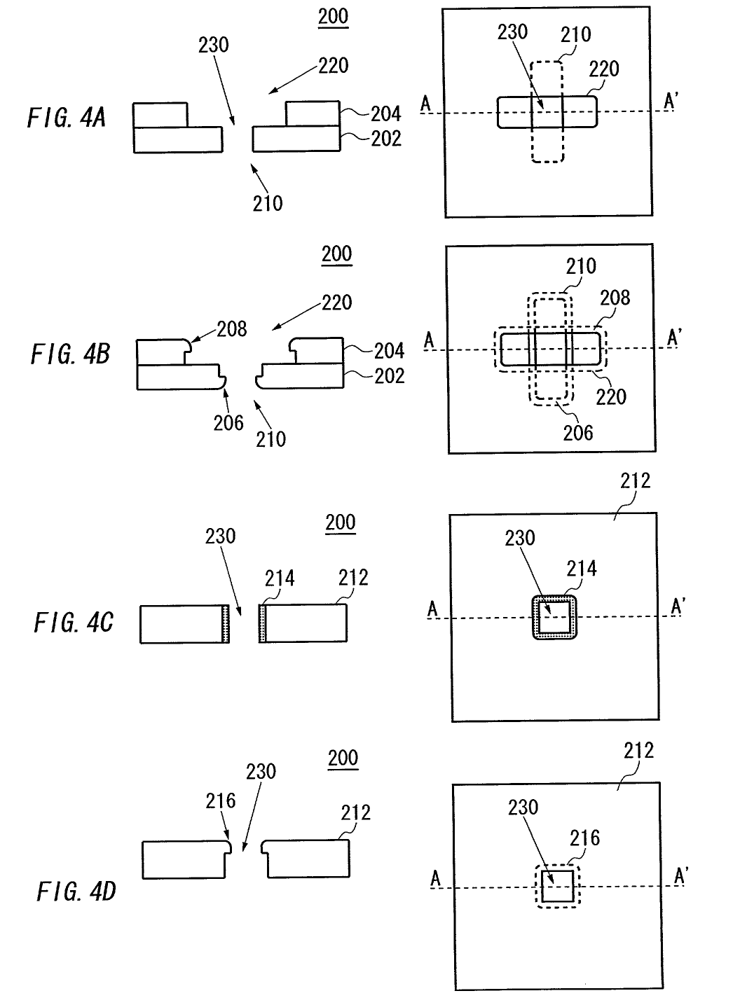

FIGS. 4(a) to 4(d) show other embodiments of the device 200 having an opening 230. In FIGS. 4(a) to 4(d), drawings on the right side are plane views of the device 200 and those on the left side are cross-sectional views seen from line A-A′ of the device 200. As shown in FIG. 4(a), the device 200 may have a first and a second layer 202 and 204. In this case, it is preferable to form the first channel 210 on the first layer 202 and the second channel 220 on the second layer 204. Further, the first and second channels 210 and 220 may be holes perforating the first and second layers 202 and 204, respectively.

As shown in FIG. 4(b), the first and second layers 202 and 204 may have protrusions 206 and 208 protruding from the first and second channels 210 and 220, respectively. The protrusions 206 and 208 may preferably be eaves or projections of the first and second layers 202 and 204, respectively. Further, it is preferable that surface coarseness of areas of the first and the second layers 202 and 204 where the protrusions 206 and 208 are formed is less than that where the protrusions 206 and 208 are not formed. For example, the first and second layers 202 and 204 are preferably formed by electrodeposition such as electroplating or electroforming.

As shown in FIG. 4(c), the device 200 maybe abase 212 having a hole through the base 212, where the base 212 includes an insert or inscribed element 214 having an opening 230 of which the vertexes are sharper than those of the hole of the base 212. The hole of the base 212 may have a first pair of substantially parallel sides and a second pair of substantially parallel sides, where the second pair of sides is substantially perpendicular to the first pair of sides. According to the present embodiment, the inscribed element 214 may be provided on a surface of the device 200 facing the electron beam generator 10 or on the opposite surface of the surface facing the electron beam generator 10. It is preferable that the vertexes of the opening 230 of the inscribed element 214 are sharper than those of the hole of the base 212.

As shown in FIG. 4(d), the device 200 maybe abase 212 having a hole and a protrusion 216 formed toward the inside of the hole, so that the hole and protrusion 216 constitute an opening 230. The hole of the base 212 may have a first pair of substantially parallel sides and a second pair of substantially parallel sides, where the second pair of sides is substantially perpendicular to the first pair of sides. It is preferable that the vertexes formed by the protrusion 216 are sharper than those of the hole of the base 212. Further, it is also preferable that the surface coarseness of the end area of the protrusion 216 is less than that of the surface of the hole of the base 212. For example, it is possible to form the surface coarseness of the end area of the protrusion 216 to be less by forming the protrusion 216 through electrodeposition of the base 212. Therefore, it is possible to form a charged particle beam of rectangular cross section having substantially right-angled vertexes by using the device 200.

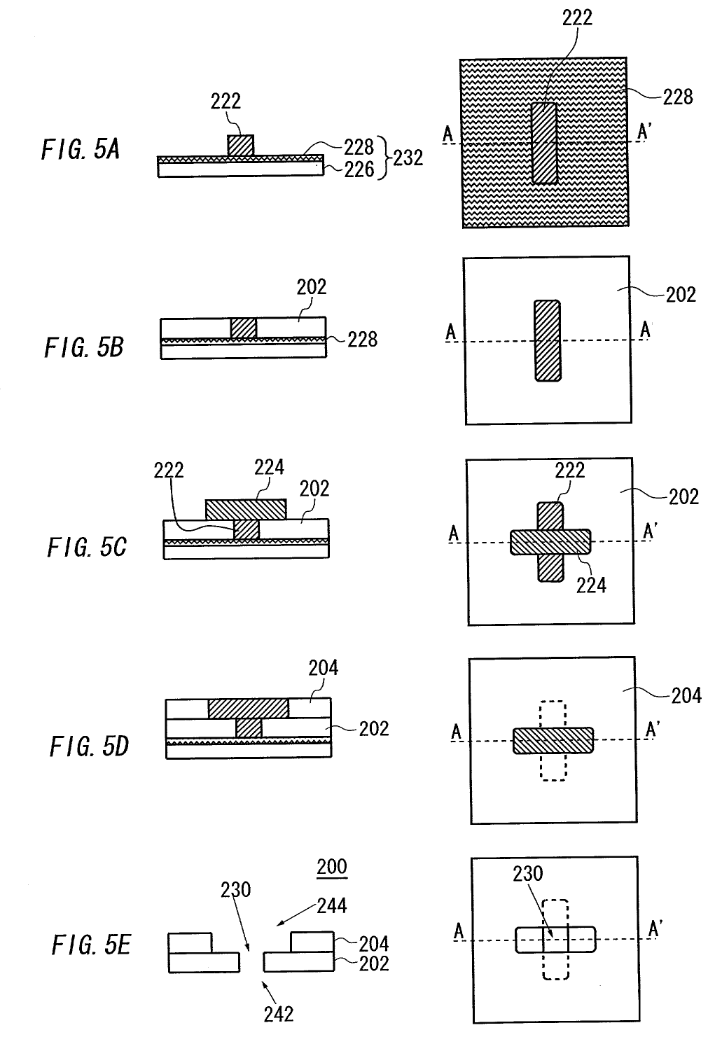

FIGS. 5(a) to 5(e) show an embodiment of a method for manufacturing the device 200 for shaping a charged particle beam according to the present invention. In FIGS. 5(a) to 5(e), drawings on the right side are plane views of the device 200 at respective process steps and those on the left side are cross-sectional views seen from line A-A′ of the device 200 at the respective process steps. First, a substrate 232 is prepared. The substrate 232 may preferably include a base 226 and a conductive film 228, where the conductive film 228 is made of a material having conductivity higher than that of the base 226. According to another embodiment of the present invention, the substrate 232 may not include the conductive film 228 by including a substrate made of a high conductive material.

FIG. 5(a) shows a process step for forming a first resist pattern 222 on an area where a first hole is formed, as described later, on the substrate 232. First, resist is coated on the substrate 232 by a spin coating method. Next, the first resist pattern 222 is formed by a photolithography process including exposure and printing processes. The first resist pattern 222 is formed to include a pair of substantially parallel sides. For the exposure process, a laser, a charged particle beam such as an electron beam or x-ray can be used as a light source. Further, the resist may preferably be selected according to the light source that is used. For example, the resist may be a positive or negative type resist, a polyimide having photosensitivity or an electron beam resist.

Further, the process step for forming the first resist pattern 222 may further include process steps for forming an intermediate layer (not shown) on the substrate 232 and etching the intermediate layer by using the first resist pattern 222 as a mask. The intermediate layer is formed between the substrate 232 and the resist pattern 222. Further, the intermediate layer may be, for example, an anti-reflection layer which decreases reflection of the light source from the substrate during the exposure process. It is preferable to perform a dry etching on the intermediate layer by using the first resist pattern 222 as a mask. Further, the process step for forming the first resist pattern 222 may be a process step for printing the first resist pattern 222.

FIG. 5(b) shows a process step for forming a first layer 202. The first layer 202 is formed on the substrate 232 by using materials selected from the group of gold (Au), platinum (Pt), copper (Cu) or nickel (Ni), etc. According to the present embodiment, the first layer 202 is selectively formed on the substrate 232 by electrodeposition. It is preferable to form the first layer 202 to have a thickness substantially the same as the thickness of the first resist pattern 222.

FIG. 5(c) shows a process step for forming a second resist pattern 224 on an area where a second hole is formed, as described later, on the first layer 202 and the first resist pattern 222. Resist is coated on the first layer 202 and the first resist pattern 222, and then the second resist pattern 224 is formed by a photolithography process including exposure and printing processes. The second resist pattern 224 is formed to include a pair of sides substantially perpendicular to and overlapped with the aforementioned substantially parallel sides of the first resist pattern 222.

FIG. 5(d) shows a process step for forming a second layer 204. The second layer 204 is formed on the first layer 202 by using materials selected from the group of gold (Au), platinum (Pt), copper (Cu) or nickel (Ni), etc. According to the present embodiment, the second layer 204 is selectively formed on the first layer 202 by electrodeposition. It is preferable to form the second layer 204 to have a thickness substantially the same as the thickness of the second resist pattern 224.

According to another embodiment of the present invention, it is also preferable to form the second layer 204 on a surface of the first layer 202 that faces the substrate 232 by turning over the first layer 202. First, after forming the first layer 202, the first layer 202 is separated from the substrate 232 by melting the conductive film 228. Next, the opposite side of a side originally contacted to the substrate 232 of the separated first layer 202 is again attached to the substrate 232. Next, resist is coated on the first layer 202 and the substrate 232, and the second resist pattern 224 is formed by a photolithography process. Then, the second layer 204 is formed on the surface of the first layer 202 that was originally contacted to the substrate 232. It is possible to effectively suppress bending of the device 200 even when the first and second layers 202 and 204 are made of materials of high internal stresses because the second layer 204 is formed on the first layer 202 by turning over the first layer 202 after forming it.

FIG. 5(e) shows a process step for separating the substrate 232. First, the first and second resist patterns 222 and 224 are removed by using, for example, resist-separating solution. Then, the device 200 is formed to include the first layer 202 having the first hole 242 with a pair of substantially parallel sides and the second layer 204 having the second hole 244 with a pair of sides substantially perpendicular to and overlapped with the aforementioned sides of the first hole 242, where the overlapped area of the first and second holes 242 and 244 forms an opening 230 which perforates the device 200. According to the present embodiment, in order to form the device 200, the conductive film 228 included in the substrate 232 is selectively melted and removed by, for example, an etchant. According to another embodiment of the present invention, the substrate 232 may be mechanically removed.

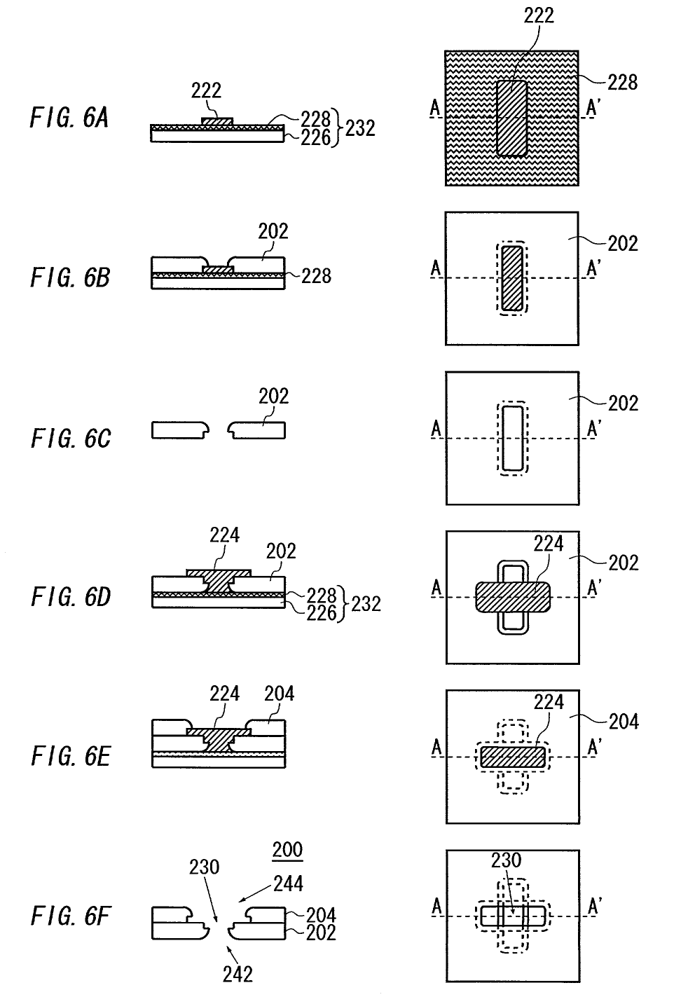

FIGS. 6(a) to 6(f) show another embodiment of the method for manufacturing the device 200 according to the present invention. In FIGS. 6(a) to 6(f), drawings on the right side are plane views of the device 200 at respective process steps and those on the left side are cross-sectional views seen from line A-A′ of the device 200 at the respective process steps. First, the substrate 232 is prepared. The substrate 232 may preferably include a base 226 and a conductive film 228, where the conductive film 228 is made of a material having conductivity higher than that of the base 226. According to another embodiment of the present invention, the substrate 232 may not include the conductive film 228 by including a substrate made of a high conductive material.

FIG. 6(a) shows a process step for forming a first resist pattern 222. Resist is coated on the substrate 232, and then the first resist pattern 222 is formed by a photolithography process including exposure and printing processes. The first resist pattern 222 is formed to include a pair of substantially parallel sides.

FIG. 6(b) shows a process step for forming a first layer 202. The first layer 202 is formed on the substrate 232 by using materials selected from the group of gold (Au), platinum (Pt), copper (Cu) or nickel (Ni), etc. It is preferable to form the first layer 202 to have a thickness substantially thicker than the thickness of the first resist pattern 222. Further, the first layer 202 is formed to cover (i.e., to “overhang”) a portion of the top surface of the first resist pattern 222 by forming the first layer 202 to be thicker than the first resist pattern 222. According to the present embodiment, the first layer 202 is selectively formed by electrode position. Further, by adjusting the process parameters of the electrodeposition, the first layer 202 is formed so that the coarseness of the surface of the first layer 202, where the first layer 202 is not contacted with the first resist pattern 222, is lower than that of the surface of the first layer 202, where the first layer 202 is contacted with the first resist pattern 222. The process parameters of the electrodeposition may include kinds, constitutions, densities, etc. of additives added to the electrodeposition solution.

FIG. 6(c) shows a process step for separating the first layer 202 from the substrate 232. First, the first resist 222 is removed. Then, the first layer 202 is separated from the substrate 232 by melting, for example, the conductive film 228.

FIG. 6(d) shows a process step for forming a second resist pattern 224. First, the opposite side of a side originally contacted to the substrate 232 of the separated first layer 202 is attached to the substrate 232. Then, resist is coated on the first layer 202 and the substrate 232, and then the second resist pattern 224 is formed by a photolithography process including exposure and printing processes. The second resist pattern 224 is formed to include a pair of sides substantially perpendicular to and overlapped with the sides of the opening that was formed in the first layer 202 when the first resist pattern 222 was removed.

FIG. 6(e) shows a process step for forming a second layer 204. The second layer 204 is formed on the first layer 202 by using materials selected from the group of gold (Au), platinum (Pt), copper (Cu) or nickel (Ni), etc. It is preferable to form the second layer 204 to have a thickness substantially thicker than the thickness of the second resist pattern 224. Further, the second layer 204 is formed to cover (i.e., to “overhang”) a portion of the top surface of the second resist pattern 224 by forming the second layer 204 to be thicker than the second resist pattern 224.

FIG. 6(f) shows a process step for separating the substrate 232. First, the second resist pattern 224 is removed by using, for example, resist-separating solution. Then, the device 200 is formed to include the first layer 202 having the first hole 242 with a pair of substantially parallel sides and the second layer 204 having the second hole 244 with a pair of sides substantially perpendicular to and overlapped with the aforementioned sides of the first hole 242, where the overlapped area of the first and second holes 242 and 244 forms an opening 230 which perforates the device 200. According to the present embodiment, in order to form the device 200, the conductive film 228 included in the substrate 232 is selectively melted and removed by, for example, an etchant.

According to the present embodiment, by respectively forming the first and second layers 202 and 204 to cover portions of top surfaces of the first and second resist patterns 222 and 224, it is possible to form the surface coarsenesses, at the surfaces for shaping charged particle beams, of the first and second layers 202 and 204 to be extremely low even when the first and second resist patterns 222 and 224 have uneven sides. Therefore, it is possible to shape the charged particle beam to have a highly precise rectangular cross section.

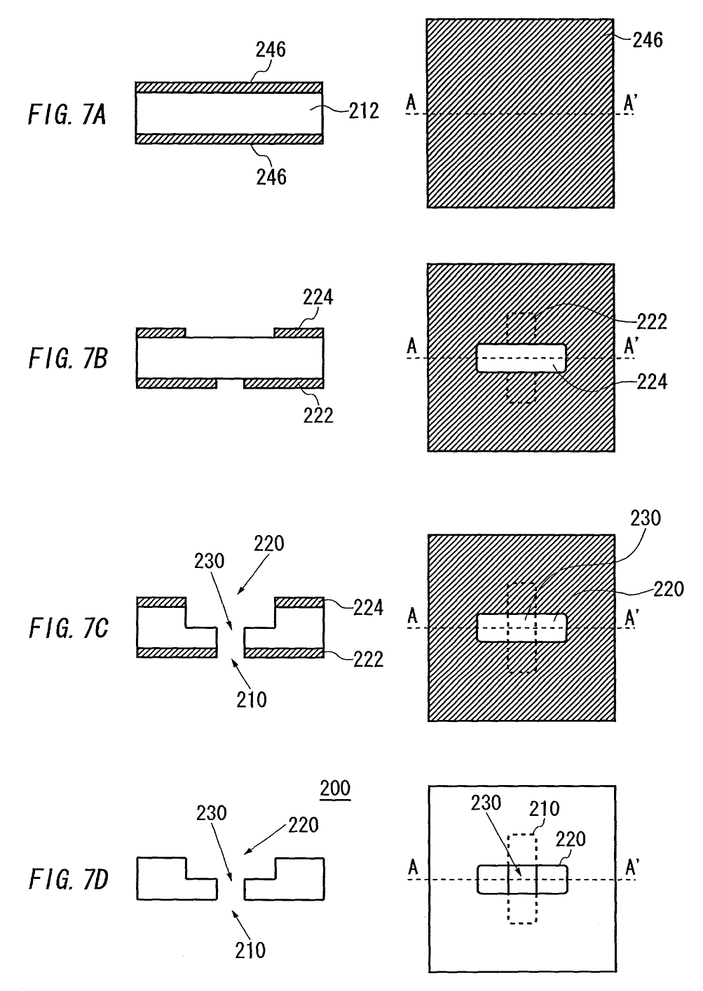

FIGS. 7(a) to 7(d) show another embodiment of the method for manufacturing the device 200 according to the present invention. In FIGS. 7(a) to 7(d), drawings on the right side are plane views of the device 200 at respective process steps and those on the left side are cross-sectional views seen from line A-A′ of the device 200 at the respective process steps.

As shown in FIG. 7(a), resist 246 is coated on both side of a substrate 212. The substrate 212 may be made of materials selected from the group of silicon (Si), silicon carbide (SiC), tungsten (W) or tantalum (Ta), etc.

FIG. 7(b) shows a process step for forming a first and a second resist pattern 222 and 224. By performing a photolithography process including exposure and printing processes, the coated resist 246 is formed to have a first resist pattern 222, which corresponds to a first channel, with a pair of substantially parallel sides, and a second resist pattern 224, which corresponds to a second channel, with a pair of sides substantially perpendicular to and overlapped with the sides of the first resist pattern 222, where the first and second channels are described below. The second resist pattern 224 is preferably formed on the opposite side of the side where the first resist pattern 222 is formed.

FIG. 7(c) shows a process step for forming the first and second channels 210 and 220. By using the first resist pattern 222 as a mask, the first channel 210 with a pair of substantially parallel sides is formed through etching the base 212. The first channel 210 is formed not to perforate the base 212. Then, by using the second resist pattern 224 as a mask, the second channel 220 with a pair of sides substantially perpendicular to and overlapped with the aforementioned sides of the first channel 210 is formed through etching the base 212, where the area defined by the overlap of the two pairs of sides of the first and second channels 210 and 220 perforates the base 212. The second channel 220 is formed so that the corresponding area of the base 212 is perforated by etching.

Then, as shown in FIG. 7(d), the first and second resist patterns 222 and 224 are removed, and the device 200 is formed to include the first channel 210 with the pair of substantially parallel sides and the second channel 220 with the pair of sides substantially perpendicular to and overlapped with the sides of the first channel 210, where the overlapped area of the first and second channels 210 and 220 forms an opening 230 which perforates the base 212.

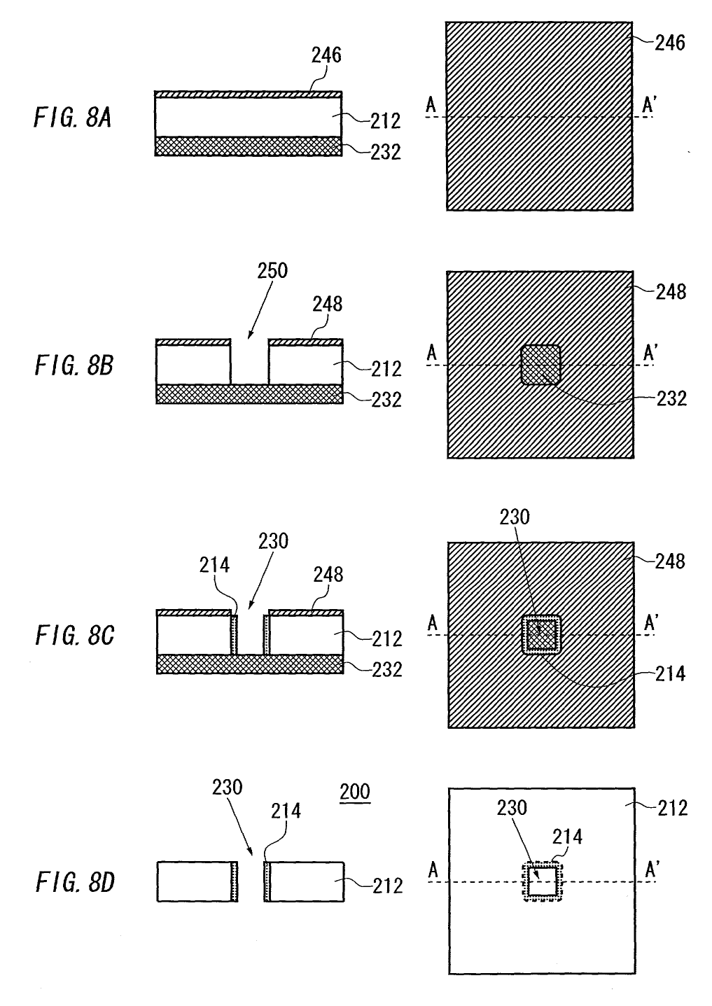

FIGS. 8(a) to 8(d) show another embodiment of the method for manufacturing the device 200 according to the present invention. In FIGS. 8(a) to 8(d), drawings on the right side are plane views of the device 200 at respective process steps and those on the left side are cross-sectional views seen from line A-A′ of the device 200 at the respective process steps.

As shown in FIG. 8(a), resist 246 is coated on a base 212 attached to a substrate 232. The substrate is preferably made of an insulating material in consideration of the following processes.

FIG. 8(b) shows a process step for forming a hole 250 having a pair of substantially parallel sides and another pair of sides substantially perpendicular to the first pair of parallel sides on the base 212. First, a resist pattern 248 is formed to have a pair of substantially parallel sides and another pair of sides substantially perpendicular to the first parallel sides on an area corresponding to the hole 250 on the base 212 by performing a photolithography process including exposure and printing processes on the resist 246. Then, the hole 250 is formed by etching the base 212 using the resist pattern 248 as a mask. It is preferable that the hole 250 be formed in the base 212 and the substrate 232 perpendicularly, and that the diameter of the hole 250 becomes smaller along the direction of etching.

FIG. 8(c) shows a process step for forming an inscribed element or insert 214. It is preferable to form the inscribed element 214 to contact with the inside surface of the hole 250. According to the present embodiment, an opening 230 is formed by the inscribed element 214 which is selectively formed on the base 212 and made of a conductive material through electroplating, where vertexes of the opening 230 are sharper than those of the hole 250.

FIG. 8(d) shows a process step for separating the substrate 232. First, the resist pattern 248 is removed. Then, by separating the substrate 232, the device 200 is completed to have the opening 230.

As apparent from the above detailed description, according to the present invention, it is possible to forma device including an opening of minute rectangular cross section with extremely high precision and ease.

Although the present invention has been described by way of exemplary embodiments, it should be understood that many changes and substitutions may be made by those skilled in the art without departing from the spirit and the scope of the present invention which is defined only by the appended claims.

What is claimed is:

1. A device comprising an opening for shaping a beam of charged particles to provide a desired cross-sectional shape thereof, comprising:
a first channel formed on a substrate of the device, the first channel including a pair of substantially parallel sides; and
a second channel formed on the substrate of the device, the second channel overlapping said first channel and including a pair of parallel sides substantially perpendicular to said pair of substantially parallel sides of said first channel,
wherein said opening perforates said substrate of the device and is formed at an overlapped area of the first and second channels.

2. A device as claimed in claim 1, wherein said second channel is formed on an opposite side of said substrate of the device than a side thereof where said first channel is formed.

3. A device as claimed in claim 1, wherein a distance between said pair of sides of said first channel is substantially the same as a distance between said pair of sides of said second channel.

4. A device as claimed in any one of claims 1, wherein more than one of said openings perforating said device are formed.

5. A device comprising an opening having a cross-sectional shape for shaping a beam of charged particles, comprising:
a base having a hole formed therein, said hole having a first pair of substantially parallel sides and a second pair of parallel sides which are substantially perpendicular to said first pair of substantially parallel sides of said hole; and
an inscribed element formed to contact with an inside surface of said hole,
wherein said inscribed element has an opening formed inside said hole, said opening of said inscribed element perforating said device and including vertexes that are sharper than corresponding vertexes of said hole.

6. A method for manufacturing a device comprising an opening for shaping a beam of charged particles to provide a desired cross-sectional shape thereof, comprising:
forming a first layer having a first hole on a substrate, said first hole having a pair of substantially parallel sides;
forming a second layer having a second hole on said first layer, said second hole having a pair of parallel sides substantially perpendicular to and overlapped with said pair of substantially parallel sides of said first hole,
wherein said opening perforates said device and is formed at an overlapped area of the first and second holes; and
separating said substrate.

7. A method for manufacturing a device as claimed in claim 6, wherein said forming said first layer comprises:
forming a first resist pattern on an area of said substrate where said first hole is formed; and
selectively forming said first layer on said substrate; and
said forming said second layer comprises:
forming a second resist pattern on an area of said first layer and said first resist pattern where said second hole is formed; and
selectively forming said second layer on said first layer.

8. A method for manufacturing a device as claimed in claim 6, wherein said substrate is made of a conductive material, and said first layer and said second layer are formed by electrodeposition.

9. A method for manufacturing a device as claimed inclaim8, wherein said first layer is formed to be thicker than said first resist pattern, and said second layer is formed to be thicker than said second resist pattern.

10. A method for manufacturing a device as claimed in claim 6, further comprising:
separating said first layer from said substrate, wherein said second layer is formed on a surface of said first layer with which said substrate originally contacted.

11. A method for manufacturing a device with an opening having a cross-sectional shape for shaping a beam of charged particles, comprising:
forming a first channel having a first pair of substantially parallel sides on a base; and
forming a second channel on said base, said second channel having a second pair of parallel sides substantially perpendicular to and overlapped with said first pair of substantially parallel sides of said first channel,
wherein said opening perforates said device and is formed at an area where the first and second channels overlap.

12. A method for manufacturing a device as claimed in claim 11, wherein said second channel is formed on a side of said base that is opposite to another side of said base on which said first channel is formed.

13. A method for manufacturing a device comprising an opening for shaping a beam of charged particles to have a selected cross-sectional shape, comprising:
forming a base having a hole formed therein, said hole being defined by a first pair of substantially parallel sides and a second pair of parallel sides substantially perpendicular to said first pair of substantially parallel sides of said hole; and
forming an inscribed element in said hole to contact with an inside surface of said hole,
wherein said inscribed element has an opening formed inside said hole, said opening of said inscribed element perforates said device and includes vertexes that are sharper than corresponding vertexes of said hole.

14. An electron beam exposure apparatus for exposing an electron beam on a desired area of a wafer, comprising:
an electron gun for generating said electron beam;
an electron lens for adjusting focus of said electron beam;
a deflector for deflecting said electron beam on a desired area of a wafer;
a device for shaping said electron beam to have a predetermined cross-sectional shape; and
a wafer stage for supporting a wafer,
wherein said device for shaping said electron beam comprises:
a first channel in a substrate of said device, said first channel having a pair of substantially parallel sides;
a second channel in said substrate of said device, said second channel having a pair of parallel sides substantially perpendicular to and overlapped with said pair of substantially parallel sides of said first channel; and
an opening which perforates said device and is formed on an area defined by said overlapped first and second channels.

15. A device for shaping a beam of charged particles, comprising:
a first channel formed in the device to have a first pair of substantially parallel sides;
a second channel formed in the device to have a second pair of substantially parallel sides, said second pair of sides of said second channel being substantially perpendicular to said first pair of sides of said first channel; and
an opening perforating the device, said opening having a substantially rectangular shape defined by an overlap of said first and second channels,
wherein a beam of charged particles is passed through said opening to provide a predetermined cross-sectional shape.
